(19) United States
(12) Patent Application Publication
(10) Pub. No.: US 2007/0250009 A1
(43) Pub. Date: Oct. 25, 2007

(54) SYRINGE PUMP

(75) Inventor: Swi Barak, Caesarea (IL)

Correspondence Address: FERENCE & ASSOCIATES LLC, 409 BROAD STREET, PITTSBURGH, PA, 15143, US

(73) Assignee: Casearea Medical Electronics, Ltd.,, Caesarea (IL)

(21) Appl. No.: 11/615,931

(22) Filed: Dec. 22, 2006

Related U.S. Application Data
(60) Provisional application No. 60/753,169, filed on Dec. 22, 2005.

(51) Int. Cl. A61M 1/00

(52) U.S. Cl. 604/151

(57) ABSTRACT

Methods and arrangements for administration of liquids to a patient by a medical pump from any size and type of syringe to a patient through a flexible tube are disclosed. Contemplated herein are methods and arrangements which permit a reduction in size of devices commonly encountered heretofore while administering liquids with high precision. While the arrangements of the present invention are of a size conducive to portability, the arrangements may also be mated with a docking station in a stationary configuration. Such a stationary configuration is preferably mounted on an IV pole.

### CROSS-REFERENCE TO RELATED APPLICATION

This application claims priority from U.S. Provisional Patent Application Ser. No. 60/753,169, filed on Dec. 22, 2005, and entitled “Syringe Pump” the contents of which are hereby fully incorporated by reference.

### FIELD OF THE INVENTION

The present invention relates generally to syringe pumps, which are useful for administration of liquids to a patient by a medical pump from any size and type of syringe to a patient through a flexible tube.

### BACKGROUND OF THE INVENTION

Syringe pumps are used to supply medication to a patient from a pre-filled syringe via a flexible tube or infusion line. Typically, a syringe pump applies a force to the plunger of the syringe to drive medication into the tube or infusion line at a controlled rate. The head of the plunger is generally engaged by a plunger head actuator that is movable along the axis of the syringe. The head actuator generally is movable from an extreme position at one end of the pump, where it allows a syringe to be loaded into the pump with its plunger fully extended, to an extreme position at the opposite end of the pump, where it fully depresses the plunger of the syringe. There are, however, a variety of different pumps available for propelling a drug to a patient. These pumps may differ—among other differences—in the manner and principle on which they operate. A need has been recognized in connection with providing arrangements and methods for reducing the size of syringe pumps without impacting the ability of the pump to administer liquids with high precision.

### SUMMARY OF THE INVENTION

The present invention, in accordance with at least one presently preferred embodiment, overcomes the disadvantages and shortcomings of previous efforts for administering a liquid to a patient in two aspects. In the first aspect, the invention is concerned with a miniature mechanism for a syringe driver, which enables the system to be portable. In the second aspect, the invention is concerned with control sensors for the use with a high precision liquid administration pump.

For a better understanding of the present invention, together with other and further features and advantages thereof, reference is made to the following description, taken in conjunction with the accompanying drawings, and the scope of the invention will be pointed out in the appended claims.

### BRIEF DESCRIPTION OF THE DRAWINGS

FIG. 1 schematically illustrates the mechanics of a syringe driver in accordance with at least one embodiment of the present invention.

FIG. 2 schematically illustrates a pump in accordance with at least one embodiment of the present invention.

### DESCRIPTION OF THE PREFERRED EMBODIMENTS

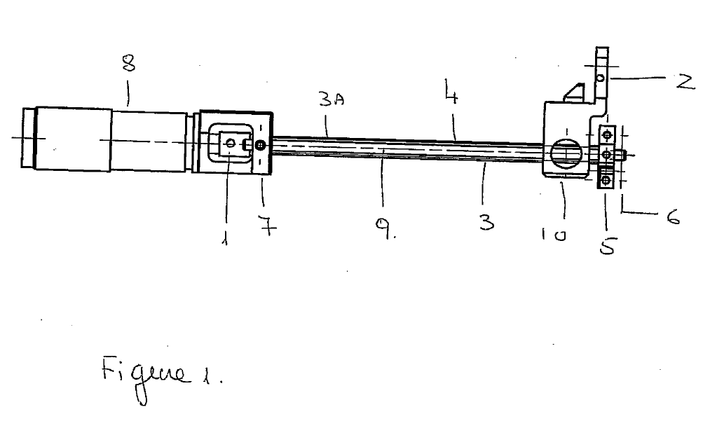

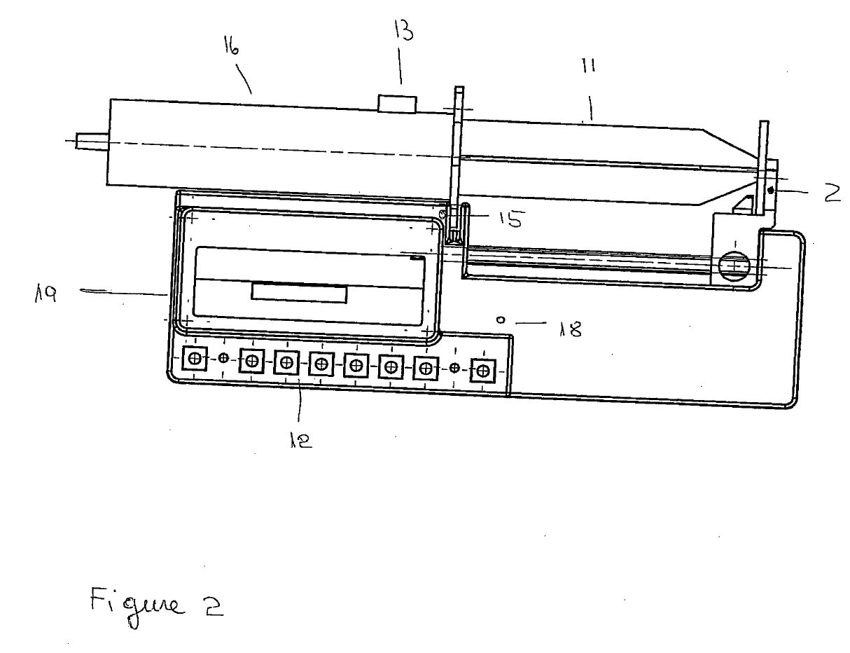

It will be readily understood that the components of the present invention, as generally described and illustrated in the Figures herein, may be arranged and designed in a wide variety of different configurations. Thus, the following more detailed description of the embodiments of the present invention, as represented in FIGS. 1 and 2, is not intended to limit the scope of the invention, as claimed, but is merely representative of selected embodiments of the invention.

Reference throughout this specification to “one embodiment” or “an embodiment” (or the like) means that a particular feature, structure, or characteristic described in connection with the embodiment is included in at least one embodiment of the present invention. Thus, appearances of the phrases “in one embodiment” or “in an embodiment” in various places throughout this specification are not necessarily all referring to the same embodiment.

Furthermore, the described features, structures, or characteristics may be combined in any suitable manner in one or more embodiments. In the following description, numerous specific details are provided to provide a thorough understanding of embodiments of the invention. One skilled in the relevant art will recognize, however, that the invention can be practiced without one or more of the specific details, or with other methods, components, materials, etc. In other instances, well-known structures, materials, or operations are not shown or described in detail to avoid obscuring aspects of the invention.

The illustrated embodiments of the invention will be best understood by reference to the drawings. The following description is intended only by way of example, and simply illustrates certain selected embodiments of apparatus and systems that are consistent with the invention as claimed herein.

In accordance with the present invention, the syringe pump mechanics preferably comprise a screw bar and two (2) guiding bars. The guiding bars preferably reduce the friction of the actuator to minimum and assure smooth operation of the mechanics. The guiding bars, however, are preferably used in parallel for data transmission wires to the pump controller (micro controller) of the data from a micro switch mounted on the actuator, if the syringe is located on the actuator slot as preferred.

Referring to FIG. 1, a preferred embodiment of the mechanics of a syringe driver in accordance with the present invention is shown. In this embodiment, a motor 8 turns the screw axis 9 that initiates, in both directions, the movement of actuator 10. The motor steps which initiate the actuator movement 10 are preferably controlled by a magnet 1 and a hall effect sensor. Two independent sensors 6 are checking as well the motor movements. The guiding bars 3 and 3A preferably act to stabilize actuator 10, during movements, under pressure, but also preferably conduct signals of sensor 2, indicating syringe location.

Referring now to FIG. 2, a pump in accordance with at least one embodiment of the present invention is shown. The pump is denoted by reference numeral 19, which is capable of being connected to a docking station (not shown). The docking station preferably has, among other features, a LED display, and the most significant data is preferably displayed on the LED display. FIG. 2 shows three sensors that are preferably used in controlling the syringe position: number 15 controls the syringe location in the syringe slot; number 13 senses the syringe diameter; and number 2 senses the syringe plunger position. An operation LED 18 preferably indicates the program status using a dual color led and number 12 depicts a key board which enables a user to set/change the programming of the pump.

In another embodiment of the present invention, the syringe pump has three sensors to detect the presence of the syringe and it's correct and safe located: a) sensor located on the actuator 2, where the signals of the sensor are transmitted through the guiding bars 3 and 3A shown on FIG. 1; b) a sensor located on the syringe location slot 15 shown on FIG. 2; and c) a third sensor located on the syringe holder 13 connected to a linear potentiometer activated by a spring (not shown) to sense the diameter of the syringe.

In another embodiment of the present invention, the pump is further provided with a covering (not shown), such as a door, which may be locked to protect any unauthorized access to the syringe. Any conventional locking mechanism may be used to secure the covering.

In another preferred embodiment, the present invention includes a motor 8 (FIG. 1) and an encoder 6, for rotating the screw axis and control the location of the actuator 10.

In a further embodiment of the present invention, there is provided a motor connected to a bearing (front bearing) 7 which is connected to a screw axis 9 and two guiding bars 3 and 3A that are connected to a second bearing (Rear Bearing) 5. The front bearing 7 has a magnet 1 on the motor axis that counts the number of motor revolutions which are compared with the results of two independent channels counted on the encoder mounted 6.

In another preferred embodiment of the present invention there is provided a motor 8 and a micro-controller, not shown, to control the motor revolution in order to get an improved linear delivery of the liquid and preventing pulsation effect. The micro-controller controls the motor revolutions by using the following algorithm:
a. the motor revolution is divided into a number of steps;
b. The micro-controller rotates the motor, sequentially from first step to the last step of each revolution, wherein each step or a group of steps are indicating the location of syringe actuator;
c. The pump output is controlled by three independent sensors which are comparing among them, results continuously; and
d. The pump can detect the syringe size used using a linear potentiometer connected to the syringe holder 13. The syringe size can be sequentially detected during the pump work or can be used for calibration to obtain the syringe size and brand.

According to another embodiment of the present invention, there is provided a dedicated flexible tube, with a pressure valve integrated, which will prevent free flow of the syringe content if pressure is not generated by the syringe actuator.

In another embodiment, the pump of the present invention may be connected to a multi-purpose docking station. Once the pump is connected to the docking station, the pump is converted into a stationary pump. The docking station preferably has a male connector—which corresponds to a female connector on the pump—and through the connector the syringe pump preferably: a) transmits the operation data to the docking station which is preferably displayed on a large and readable display; b) charges the pump's rechargeable batteries and will supply DC to the pump, while connected to electrical mains; c) transmits data to a central computer memory to create a file that can be tracked down; and d) enables connectivity to a central computer for data setting and control parameters.

Although illustrative embodiments of the present invention have been described herein with reference to the accompanying drawings, it is to be understood that the invention is not limited to those precise embodiments, and that various other changes and modifications may be affected therein by one skilled in the art without departing from the scope or spirit of the invention.

What is claimed is:

1. A miniature syringe pump and a docking station mounted on an iv pole.

2. The docking station that receive data from the, pump through a corresponding connector and displays it on a large visible, from distance, display.

3. The docking station that receive data from the Pump which has an LCD display, through a corresponding connector and converts the data and displays it on a LED display.

4. A docking station that receive data from a portable pump, of any type and kind, including infusion pumps, having an LCD (LIQUID CRYSTAL DISPLAY) through a corresponding connector, converts the data and displays it on a LED display.

5. A docking station that enable data collection from the pump.

6. A miniature mechanism that enable data transmission from a movable part through 2 guiding bars.

7. Disposable administration set with a pressure and one-way valve integrated.

8. A syringe pump with 3 independent sensors that compares motor revolutions.
